(19) United States
(12) Patent Application Publication
(10) Pub. No.: US 2021/0386676 A1
(43) Pub. Date: Dec. 16, 2021

(54) METHOD OF CHEMICAL TAGGING OF TABLETS USING THE COATING

(71) Applicant: INSTYTUT BIOTECHNOLOGII I MEDYCYNY MOLEKULARNEJ, Gdansk (PL)

(72) Inventors: Rafal BANASIUK, Gdansk (PL); Dawid NIDZWORSKI, Gdansk (PL); Mateusz PODDEBNIAK, Warszawa (PL); Kinga SIATKOWSKA, Gdansk (PL)

(73) Assignee: INSTYTUT BIOTECHNOLOGII I MEDYCYNY MOLEKULARNEJ, Gdansk (PL)

(21) Appl. No.: 17/284,878

(22) PCT Filed: Oct. 16, 2019
(86) PCT No.: PCT/PL2019/000094

§ 371 Date: Apr. 13, 2021

(30) Foreign Application Priority Data
Oct. 16, 2018 (PL) P.427437

(51) Int. Cl. A61K 9/28

(52) U.S. Cl. CPC A61K 9/282

(57) ABSTRACT

A method of chemically tagging tablet coatings and readout of the tag includes addition of the specific mass of the tagging substance to the coating and preparation of coatings with the tagging substance. The method additionally includes extraction of the compounds present in the tablet coating and chromatographic analysis of the compounds present in the coating. Further, the method includes comparison of the absolute, non-unitary concentration values and paraben mass to authenticate the results.

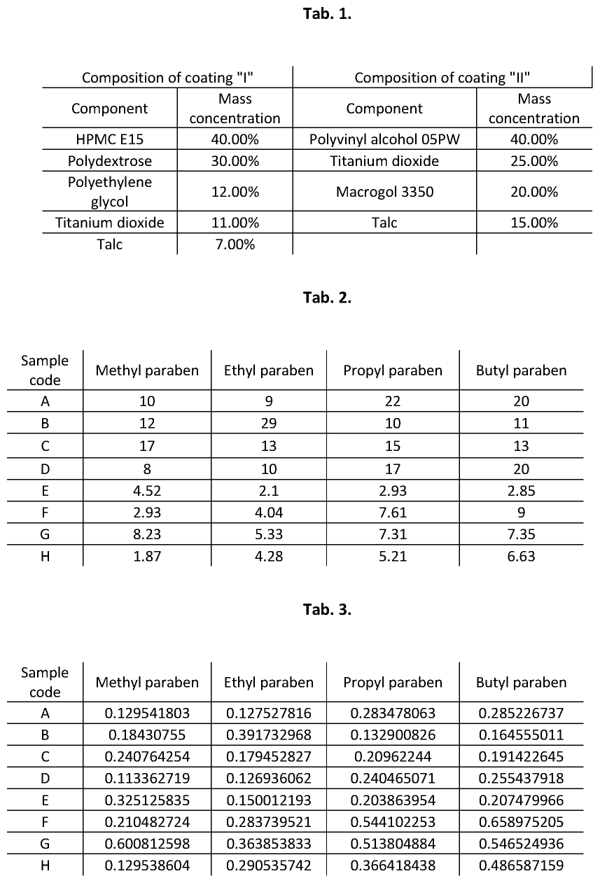

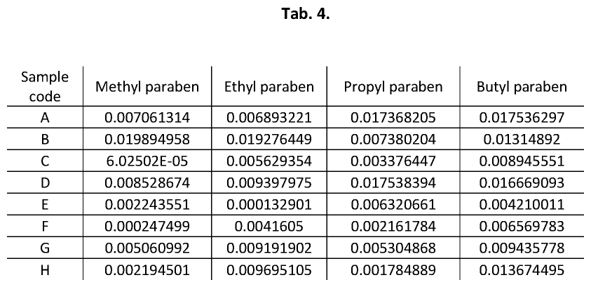

The subject of the invention is the method of chemical tagging, reading the chemical marker, and reading the authentication of the code generated using changes in the concentration of the tagging substances in the tablet coating.

Counterfeit medicine is a severe problem in the pharmaceutical industry. Both brand and generic medicines, as well as prescription and non-prescription medications, are the subject of counterfeiting. An average consumer is unable to differentiate the authentic and counterfeit medicine—both the packaging and the medicine form seem to be correct, while the difference lies in the form or dose of the active substance, and the presence of harmful substances. According to the Pharmaceutical Security Institute, over half a thousand counterfeit versions of pharmaceuticals, ranging from painkillers to antibiotics and anticancer drugs, are present on the global market. This problem affects at least 123 countries. Most of the counterfeit medicine is entering the legal drug supply chain are forged live-saving medicines.

According to the World Health Organization, over a million people globally die every year because of counterfeit medicine. 15% drugs on the global market are counterfeit. However, the problem affects mainly some regions of Asia, Africa, and South America where the percentage can reach even 70% in the developing countries.

According to the European Parliament and Council Directive 2011/62/EU from Jun. 8, 2011, a pan-European computer system that will enable the verification of the medicine authenticity based on the unique packaging codes will start operating in 2019. This applies to all prescription drugs and only certain non-prescription medicines. A pharmacist will scan the codes before the drug is released to the patient. Therefore, many companies have been established which offer labelling of the packagings with numerical codes, bar codes, or QR codes that can be scanned using mobile applications or special scanners. Labelling of the packaging does not solve the problem of the counterfeit products. Technologies enabling the direct identification of the drugs are required.

The approach proposed in the present invention offers multilevel protection of the product authenticity based on the conformity of the codes on the packagings with the unique chemical code obtained by the addition of the combination of the compounds in a specific proportion. It is a simple and economically advantageous method that does not require expensive and sophisticated technologies. Tablets authentication is conducted in laboratories ensuring high accuracy and reliability of the results.

The chemical tag consists of at least two compounds to obtain the proportion. In consequence, the tag with the serial code present on the packaging gives a unique identification for the tablets lot. The invention provides the variability of codes for each manufacturing batch.

The compounds which are not present in the drug coating or are present in a constant value can be used for the tagging. Small-molecule compounds with good UV absorption are preferred.

The subject of the present invention is the method of medicine chemical tagging using a coating, comprising of:
Addition of the known amount of labelling compounds to the tablet coating in the concentration not higher than 10%
Extraction of the compounds from the coating using an organic solvent
Chromatographic analysis of the coating compounds
Comparative analysis of the coating compounds against chemical standards Addition of the labelling substances does not alter the pharmacokinetics or pharmacodynamics of the medicine. The used concentrations of the labelling substances are neutral for the human organism. Addition of the labelling substance does not alter the physical appearance of the tablet.

The subject of the present invention is the method of the chemical tagging of tablets where the marker is defined as a mutual, absolute, and non-unitary ratio of the labelling substances.

The method in which parabens can be used as the labelling substances.

The method which can be used for any type of the coating.

The method in which the addition of the labelling substance does not change the pharmacokinetic and pharmacodynamic properties of the coated substance.

The method in which the difference of the absolute ratio between normalised concentration value and normalised mass of the added substances cannot exceed 0.02 for the correct reading.

### TABLE DESCRIPTION

Tab. 1—Coating composition given in the compound mass percentage

Tab. 2—Mass of the parabens used for the experiment given in milligrams

Tab. 3—Concentration of parabens obtained as a result of the chromatographic analyses given in milligrams per millilitre

Tab. 4—Difference between the expected value and the obtained value of the normalised results (absolute, nonunitary value)

The invention is illustrated by the following embodiments, including but not limited to.

### EXAMPLE 1

1. 5 g of the coating “I” was weighed four times using an analytical weight.
2. The concentration of specific parabens (A-D) was added to each weighed amount according to Tab. 2.
3. The samples with parabens were thoroughly homogenised
4. The prepared coating was used to prepare tablets
5. The coating was scraped from the tablets in the amount of 0.5 g per sample
6. The scraped coating was extracted by vigorous shaking in 5 mL of acetonitrile for 15 minutes
7. The sample was centrifuged, filtered through the Teflon syringe filter and transferred to the chromatographic vial
8. High-performance liquid chromatography was used for the analysis of the paraben concentrations in the sample—operational parameters: C18 column, 50×4.6 mm, 1.8 μm bead, isocratic flow (acetonitrile:water—30:70), 1 mL/min; sample volume: 5 μL, analysis time: 12 minutes.
9. The result was used to calculate the concentration in the sample based on five-point calibration curves for each tested compound.
10. The obtained results were normalised according to the formula:

(concentration of the selected compound)/(sum of the concentrations of all compounds determined in the sample)
11. The concentrations of the added compounds were normalised according to the formula:

(mass of the selected compound)/(mass of all compounds added to the sample)
12. After normalisation, the results were compared by subtraction of the normalised value of the obtained concentration from the normalised mass value of the added compounds.
13. If the result lower than the absolute value of 0.02 is obtained, the result is considered correct, and the code identity is confirmed.

### EXAMPLE 2

1. 1 g of the coating “II” was weighed four times using an analytical weight.
2. The concentration of specific parabens (E-I) was added to each weighed amount according to Tab. 2.
3. The samples with parabens were thoroughly homogenised
4. The prepared coating was used to prepare tablets
5. The coating was scraped from the tablets in the amount of 0.5 g per sample
6. The scraped coating was extracted by vigorous shaking in 5 mL of acetonitrile for 15 minutes
7. The sample was centrifuged, filtered through the Teflon syringe filter and transferred to the chromatographic vial
8. High-performance liquid chromatography was used for the analysis of the paraben concentrations in the sample—operational parameters: C18 column, 50×4.6 mm, 1.8 μm bead, isocratic flow (acetonitrile:water—30:70), 1 mL/min; sample volume: 5 μL, analysis time: 12 minutes.
9. The result was used to calculate the concentration in the sample based on five-point calibration curves for each tested compound.
10. The obtained results were normalised according to the formula:

(concentration of the selected compound)/(sum of the concentrations of all compounds determined in the sample)
11. The concentrations of the added compounds were normalised according to the formula:

(mass of the selected compound)/(mass of all compounds added to the sample)
12. After normalisation, the results were compared by subtraction of the normalised value of the obtained concentration from the normalised mass value of the added compounds.
13. If the result lower than the absolute value of 0.02 is obtained, the result is considered correct, and the code identity is confirmed.

The tagging method can be used universally, regardless of the coating composition and the coated active substance. Thanks to the easy analytical method, it is possible to verify the authenticity of the tablet in most of the analytical laboratories.

1. A method of chemical tagging of tablets, the method comprising:
providing a tagging substance;
preparing a coating for the tablets including the tagging substance; and
applying the coating to the tablets, wherein the tagging substance includes a tag defined as reciprocal, absolute, and nonunitary ratio of the tagging substances.

2. The method according to the claim 1, wherein the tagging substance includes parabens.

3. The method according to the claim 1, wherein the method is used for any type of coating.

4. The method according to the claim 1, wherein the tagging substance does not change pharmacokinetic and pharmacodynamic properties of the coating.

5. The method according to the claim 1, wherein an absolute value of a ratio between a normalised concentration and a normalised mass of added tagging substances is no greater than 0.02.

6. A method of chemically tagging tablet coatings and readout of the tag, the method comprising:
adding a specific mass of a tagging substance to the coating;
preparing coatings with the tagging substance;
extracting compounds present in the tablet coating;
chromatographic analysis of the compounds present in the coating; and
comparing absolute, non-unitary concentration values and paraben mass to authenticate the results.

7. A tablet comprising:
a tablet coating having a specific mass of a tagging substance,
wherein the tagging substance includes a tag defined as reciprocal, absolute, and nonunitary ratio of the tagging substances.
